(19) United States
(12) Patent Application Publication
(10) Pub. No.: US 2021/0378974 A1
(43) Pub. Date: Dec. 9, 2021

(54) LIPID COMPOSITION FOR ENCAPSULATING AN ACTIVE SUBSTANCE, PERMITTING CONTROL OF THE RATE OF RELEASE OF SAID ACTIVE SUBSTANCE

(71) Applicant: SOCIETE D'EXPLOITATION DE PRODUITS POUR LES INDUSTRIES CHIMIQUES SEPPIC, PARIS CEDEX 07 (FR)

(72) Inventors: Sophie FAGET, CASTRES (FR); Sandra LEFEBVRE, CASTRES (FR)

(21) Appl. No.: 17/286,135

(22) PCT Filed: Oct. 16, 2019
(86) PCT No.: PCT/FR2019/052454

§ 371 Date: Apr. 16, 2021

(30) Foreign Application Priority Data
Oct. 19, 2018 (FR) 1859696

(51) Int. Cl. A61K 9/50; A61K 47/26; A23K 20/158; A23K 10/20; A23L 33/12; A23P 10/35

(52) U.S. Cl. CPC A61K 9/5063; A61K 47/26; A61K 9/5089; A23V 2002/00; A23K 10/20; A23L 33/12; A23P 10/35; A23K 20/158

(57) ABSTRACT

Disclosed is a lipid composition including, per 100% of its weight:—from 40% to 99.9% by weight of a component including, per 100% of its weight, from 90% to 100% by weight of beeswax and up to 10% by weight of at least one other lipid excipient,—from 0.1% to 60% by weight of at least one lipophilic surfactant.

### CROSS-REFERENCE TO RELATED APPLICATIONS

This application is the U.S. national phase of International Application No. PCT/FR2019/052454 filed Oct. 16, 2019 which designated the U.S. and claims priority to FR 1859696 filed Oct. 19, 2018, the entire contents of each of which are hereby incorporated by reference.

### BACKGROUND OF THE INVENTION

### Field of the Invention

The present invention relates to a lipid composition, to a controlled-release composition comprising said lipid composition and enabling control of the rate of release of the active substance it contains, and to a process for manufacturing the galenical formulation comprising the controlled-release composition.

### Description of the Related Art

The active principles developed and used in human and veterinary pharmaceutical formulations and in formulations from the food supplement industries are sensitive to environmental factors, whether during the manufacture of the associated galenical forms (in particular to the elevated temperatures used in certain processes, to oxidation phenomena, etc.) and/or during the lifetime of said galenical forms, and/or during consumption, in the human or animal body, when said galenical forms are contacted with the degradation and/or digestion molecules present in the bodies of the consumers.

In addition, the molecules making up all or some of the active principles present in these galenical forms may have organoleptic characteristics which are incompatible with direct consumption: a bad taste, inappropriate odor, etc.

To provide the nutritional or therapeutic advantages aimed for, the molecules introduced into capsules, hard capsules, tablets or even into food should not dissolve at the same rate and in the same environment.

It is in particular for these various reasons that many active molecules are involved in a process referred to as encapsulation, the objective of which is to prepare a composition (C_{A}) comprising at least one active principle. This encapsulation process has the function of protecting the at least one active principle from undesired interactions with a specific external environment, and of transporting said active principle(s) into another environment in which the conditions allow for release of the active agent in order to accomplish its intended therapeutic or nutritional function. This process is a particular example of the process called “direct functionalization” by those skilled in the art.

The term “direct functionalization” is understood to mean the modification of at least one physicochemical property of the active pharmaceutical ingredient by the implementation of a particular formulation and by means of specific processes.

Among the best-known encapsulation technologies, mention may be made of the technologies employing predominantly hydrophilic compounds but also technologies employing predominantly hydrophobic compounds, for example the technologies of “prilling”, that is to say technologies comprising a step of atomization of a lipid form containing at least one active agent so as to obtain a solid spherical form, also called “spray chilling” or “spray cooling”, of congealing, of hot melt coating, of hot melt extrusion, of melt granulation, of pelletization, of spheronization, of thermogranulation, etc.

This “direct functionalization”, in particular by means of an encapsulation technique, can thus present the advantages of stabilization of certain compounds that are intrinsically unstable, such as for example volatile compounds, of prevention of oxidation phenomena, and of overall protection of the drug or nutritional activity.

This step of initial “direct functionalization”, in particular by means of an encapsulation technique, can also have an impact on the kinetics of release of the active agent in the host organism. This effect is often sought after, in particular with the aim of masking taste, odor or in order to obtain delayed, controlled release of the active agent at the targeted biological zone in the human or animal body.

The impact of this “direct functionalization” step on the release profile of the active agent is taken into account during the phases of development of a therapeutic or prophylactic or nutritional composition.

However, during scale-up phases, in the course of which industrial forming tools are used, said tools involving mechanical stresses on the galenical form, those skilled in the art find themselves confronted with the recurring problem of an undesired change in the controlled release profile compared to the formulation initially tested on scales not requiring the use of such tools.

By way of example, the release profile of an active agent after “direct functionalization” may be drastically modified, accelerated or slowed, following use in a process inducing significant mechanical stress such as a compression process. It is thus possible for a lipid protection induced by the initial “functionalization” to be impaired by weakening the “protective shell” during the compression, which may induce an accelerated release profile of the active agent.

Over the course of the downstream steps of galenically forming these prolonged release formulations, it was observed that the release profile of the active agent obtained and demonstrated after functionalization can be significantly affected and can show a significant difference with respect to the release profile of the active agent before galenical forming. Thus, the release profile of an active agent after functionalization may be drastically modified, accelerated or slowed, following the implementation of a step involving significant mechanical stress in a process for preparing said functionalized active agent, such as for example a compression step. More particularly by way of example, a lipid protection induced by the initial functionalization may be impaired by weakening the “protective shell” during the compression, which can lead to an accelerated and undesired release profile of the active agent.

To date and to the applicant's knowledge, those skilled in the art do not possess a solution making it possible to develop and to manufacture formulations comprising an active principle which is to be released in a delayed or prolonged fashion, and the release profile of which is not altered by a preparation process comprising at least one step implementing significant mechanical stress on said formulation and/or on a galenical form prepared by a process comprising at least one step employing significant mechanical stress.

### SUMMARY OF THE INVENTION

A solution of the present invention is a lipid composition comprising, per 100% of its weight:

from 40% to 99.9% by weight, more particularly from 40% to 98%, and more particularly still from 40% to 95% by weight, of a component comprising, per 100% of its weight, from 90% to 100% by weight of beeswax and up to 10% by weight of at least one other lipid excipient,

from 0.1% to 60% by weight, more particularly from 2% to 60% by weight, and more particularly still from 5% to 60% by weight, of at least one lipophilic surfactant selected from polyethoxylated fatty acids, esters of fatty diacids and of polyethylene glycols, esters of polyglycerol and of fatty acids, esters of propylene glycol and of fatty acids, mixtures of esters of propylene glycol and of esters of glycerol, fatty acid diglycerides, sterols and derivatives of sterol, esters of fatty acids and of sorbitan, esters of sorbitan, of polyethylene glycols and of fatty acids, ethers of polyethylene glycols and of alkyl, sucrose esters, and polyoxyethylene-polyoxypropylene block copolymers.

The composition according to the invention can take various forms which are solid at ambient temperature (beads, spheres, scales, flakes, pearls, etc.). It is principally intended for the forming of active agents in the animal or human pharmaceutical, therapeutic and/or prophylactic industrial sectors, the food supplement and/or human and animal food industries.

For the purposes of the present invention, the term “lipid excipient” is understood to mean an excipient having a melting point of less than or equal to 120° C., preferentially of less than or equal to 100° C., which is solid at ambient temperature (greater than or equal to 15° C. and less than or equal to 30° C.), and which is insoluble or sparingly soluble in water.

The beeswax used in the composition according to the invention is yellow or white, is also denoted by the number E901 and has a melting point of between 60 and 67° C.

Regarding the lipophilic surfactant, it is specified here that the terms “lipophilic” and “hydrophilic” are relative terms. An empirical parameter commonly used to characterize the relative lipophilicity and hydrophilicity of non-ionic amphiphilic compounds is the hydrophilic-lipophilic balance, that is to say the value known as “HLB”.

Thus, surfactants having lower HLB values are more lipophilic and have a greater solubility in oils, whereas surfactants having higher HLB values are more hydrophilic and have a greater solubility in aqueous solutions.

Using the HLB values as an indication, hydrophilic surfactants are generally considered as being compounds having an HLB value of greater than or equal to 10, and also anionic, cationic or zwitterionic compounds for which the HLB scale is generally not applicable. Likewise, lipophilic surfactants are compounds having an HLB value of less than approximately 10. In both cases, the term “approximately” is mentioned due to induced variability.

Depending on the case, the lipid composition according to the invention may have one or more of the features below:

the lipid excipient is selected from animal waxes, vegetable waxes, mineral waxes, synthetic waxes or hydrogenated vegetable oils;

the lipophilic surfactant is selected from esters of fatty acids and of sugars;

the lipophilic surfactant is a lipophilic surfactant from the family of the sorbitan esters, more particularly an element from the group consisting of sorbitan monolaurate, sorbitan monopalmitate, sorbitan monostearate and sorbitan monooleate, and even more particularly sorbitan monopalmitate or sorbitan monostearate, and even more particularly sorbitan monostearate;

said composition comprises from 0 to 20% by weight of one or more hydrophilic surfactants, more particularly from 0% to 10% by weight;

the hydrophilic surfactant or surfactants is/are selected from soya lecithin, polyethoxylated sorbitan esters, polyethoxylated alcohols, polyethoxylated acids, polyglycerol esters, glucose ethers and block copolymers of ethylene oxide and of propylene oxide;

the lipophilic surfactant is a sorbitan ester and the hydrophilic surfactant is selected from polyethoxylated sorbitan esters;

said composition comprises: 80% beeswax, 20% sorbitan stearate;

said composition comprises: 94% beeswax, 1% sorbitan stearate, and 5% sorbitan oleate polyethoxylated with 20 moles of ethylene oxide (also called polysorbate 80);

said composition comprises: 75% beeswax, 20% sorbitan stearate, and 5% sorbitan oleate polyethoxylated with 20 moles of ethylene oxide (also called polysorbate 80);

said composition comprises: 50% beeswax, 45% sorbitan stearate, and 5% sorbitan oleate polyethoxylated with 20 moles of ethylene oxide (or polysorbate 80);

said composition comprises from 0 to 20% by weight, and more particularly from 0 to 10% by weight, of at least one coating adjuvant;

the coating adjuvant or adjuvants is/are selected from diluents, flavorings, appetizing agents, colorants, antioxidants, plasticizers, antifoaming agents and disintegrants.

Regarding the lipid excipient:

for animal waxes, mention may be made of: spermaceti which has a melting point of between 52 and 55° C., lanolin which has a melting point between 37 and 44° C., and shellac which has a melting point of between 77 and 90° C.;

for vegetable waxes, mention may be made of: carnauba wax which has a melting point of between 78 and 88° C., candelilla wax which has a melting point of between 67-79° C., and rice bran wax which has a melting point of close to 78° C.;

for mineral waxes, mention may be made of: paraffin which has a melting point of between 50 and 71° C., and microcrystalline wax which has a melting point of between 54 and 102° C.;

for synthetic waxes, mention may be made of: Fischer-Tropsch waxes, polyethylene (or polypropylene) waxes, poly(ethylene oxide) or poly(propylene oxide) waxes, etc.;

for hydrogenated vegetable oils, mention may be made of: palm oil which has a melting point of between 58 and 62° C., and stearin which has a melting point of between 61 and 65° C.

Among the lipophilic surfactants, mention may be made of:

esters of fatty acids and of glycerol, said fatty acids being selected from stearic, palmitic, ketostearic, arachidic and behenic acids;

ethers of fatty alcohol and of sugars, the fatty alcohols being stearic, palmitic, ketostearic, arachidic and behenic alcohols; said sugars being for example reducing sugars and more particularly glucose, xylose, arabinose, mannose or sucrose;

divalent salts of fatty acids, such as the magnesium, zinc or calcium salts of stearic, palmitic, ketostearic, arachidic and behenic acids;

fatty alcohols condensed with propylene oxide and/or butylene oxide;

block copolymers of alkoxides (ethylene, propylene, butylene, etc.), which are rich in propylene or butylene oxide;

esters of fatty acids and of sugars; said fatty acids been selected from stearic, palmitic, ketostearic, arachidic and behenic acids; said sugars being for example glucose, sorbitol, mannitose, mannitol, sucrose, mannose, xylitol or xylose;

among the esters of fatty acids and of sugars, mention may in particular be made of esters of fatty acids and of sorbitol, and esters of fatty acids and of sorbitan.

Among the sorbitan esters which can be combined with the composition which is a subject matter of the present invention, mention may be made of:
sorbitan monolaurate, sold by SEPPIC under the brand name Montane™ 20, by Croda under the brand name Span′ 20 and by,
sorbitan monopalmitate, sold by SEPPIC under the brand name Montane™ 40 and by Croda under the brand name Span′ 40,
sorbitan monostearate, sold by SEPPIC under the brand name Montane™ 60 and by Croda under the brand name Span′ 60,
sorbitan monooleate, sold by SEPPIC under the brand name Montane™ 80 and by Croda under the brand name Span™ 80,
sorbitan trioleate, sold by SEPPIC under the brand name Montane™ 85 and by Croda under the brand name Span™ 85,
sorbitan sesquioleate, sold by SEPPIC under the brand name Montane™ 83, by Croda under the brand name Crill™ 43 and by,
sorbitan tristearate, sold by SEPPIC under the brand name Montane™ 65 and by Croda under the brand name Span™ 65,
sorbitan monoisostearate, sold by SEPPIC under the brand name Montane™ 70 and by Croda under the brand name Crill™ 6.

The composition according to the invention optionally comprises from 0 to 20% of one or more hydrophilic surfactants selected from soya lecithin, ethoxylated sorbitan esters, polyethoxylated alcohols, polyethoxylated acids, polyglycerol esters, glucose ethers and block copolymers of ethylene oxide and of propylene oxide.

Among the ethoxylated sorbitan esters which can be combined with the composition which is a subject matter of the present invention, mention may be made of:

sorbitan monolaurate ethoxylated with 20 moles of ethylene oxide, sold by SEPPIC under the brand name Montanox™ 20 and by Croda under the brand name Tween™ 20,

sorbitan monolaurate ethoxylated with 4 moles of ethylene oxide, sold under the brand name Tween™ 21,

sorbitan monolaurate ethoxylated with 6 moles of ethylene oxide, sold under the brand name Nikkol™ GL-I by Nikko,

sorbitan monopalmitate ethoxylated with 20 moles of ethylene oxide, sold by SEPPIC under the brand name Montanox™ 40 and by Croda under the brand name Tween™ 40,

sorbitan monostearate ethoxylated with 20 moles of ethylene oxide, sold by SEPPIC under the brand name Montanox™ 60 and by Croda under the brand name Tween™ 60,

sorbitan tristearate ethoxylated with 20 moles of ethylene oxide, sold by SEPPIC under the brand name Montanox™ 65 and by Croda under the brand name Tween™ 65,

sorbitan monooleate ethoxylated with 20 moles of ethylene oxide, sold by SEPPIC under the brand name Montanox™ 80 and by Croda under the brand name Tween™ 80,

sorbitan trioleate ethoxylated with 20 moles of ethylene oxide, sold by SEPPIC under the brand name Montanox™ 85 and by Croda under the brand name Tween™ 85,

sorbitan monoisostearate ethoxylated with 20 moles of ethylene oxide, sold by SEPPIC under the brand name Montanox™ 70 and by Croda under the brand name Tween™ 120,

sorbitan monostearate ethoxylated with 4 moles of ethylene oxide, sold by Croda under the brand name Tween™ 61,

sorbitan monooleate ethoxylated with 5 moles of ethylene oxide, sold by SEPPIC under the brand name Montanox™ 81 and by Croda under the brand name Tween™ 81.

Among the ethoxylated fatty alcohols which can be combined with the composition which is a subject matter of the present invention as hydrophilic surfactants, mention may be made of oleyl alcohol ethoxylated with 2 moles of ethylene oxide, oleyl alcohol ethoxylated with 3 moles of ethylene oxide, oleyl alcohol ethoxylated with 5 moles of ethylene oxide, oleyl alcohol ethoxylated with 10 moles of ethylene oxide, oleyl alcohol ethoxylated with 20 moles of ethylene oxide, lauryl alcohol ethoxylated with 4 moles of ethylene oxide, lauryl alcohol ethoxylated with 7 moles of ethylene oxide, lauryl alcohol ethoxylated with 9 moles of ethylene oxide, lauryl alcohol ethoxylated with 23 moles of ethylene oxide, cetyl alcohol ethoxylated with 2 moles of ethylene oxide, cetyl alcohol ethoxylated with 10 moles of ethylene oxide, cetyl alcohol ethoxylated with 20 moles of ethylene oxide, stearyl alcohol ethoxylated with 2 moles of ethylene oxide, stearyl alcohol ethoxylated with 10 moles of ethylene oxide, stearyl alcohol ethoxylated with 20 moles of ethylene oxide or stearyl alcohol ethoxylated with 100 moles of ethylene oxide.

Among the ethoxylated fatty acids which can be combined with the composition which is a subject matter of the present invention as hydrophilic surfactants, mention may be made of monolauric acid ethoxylated with between 4 and 200 moles of ethylene oxide, and more particularly monolauric acid ethoxylated with 4 moles of ethylene oxide, monolauric acid ethoxylated with 6 moles of ethylene oxide, monolauric acid ethoxylated with 7 moles of ethylene oxide, monolauric acid ethoxylated with 8 moles of ethylene oxide, monolauric acid ethoxylated with 10 moles of ethylene oxide, monolauric acid ethoxylated with 50 moles of ethylene oxide, monolauric acid ethoxylated with 100 moles of ethylene oxide or monolauric acid ethoxylated with 200 moles of ethylene oxide, monooleic acid ethoxylated with between 4 and 200 moles of ethylene oxide, and more particularly monooleic acid ethoxylated with 1 mole of ethylene oxide, monooleic acid ethoxylated with 2 moles of ethylene oxide, monooleic acid ethoxylated with 4 moles of ethylene oxide, monooleic acid ethoxylated with 5 moles of ethylene oxide, monooleic acid ethoxylated with 6 moles of ethylene oxide, monooleic acid ethoxylated with 8 moles of ethylene oxide, monooleic acid ethoxylated with 9 moles of ethylene oxide, monooleic acid ethoxylated with 10 moles of ethylene oxide, monooleic acid ethoxylated with 50 moles of ethylene oxide, monooleic acid ethoxylated with 100 moles of ethylene oxide or monooleic acid ethoxylated with 200 moles of ethylene oxide, monostearic acid ethoxylated with between 4 and 200 moles of ethylene oxide, and more particularly monostearic acid ethoxylated with 1 mole of ethylene oxide, monostearic acid ethoxylated with 2 moles of ethylene oxide, monostearic acid ethoxylated with 4 moles of ethylene oxide, monostearic acid ethoxylated with 5 moles of ethylene oxide, monostearic acid ethoxylated with 6 moles of ethylene oxide, monostearic acid ethoxylated with 8 moles of ethylene oxide, monostearic acid ethoxylated with 9 moles of ethylene oxide, monostearic acid ethoxylated with 10 moles of ethylene oxide, monostearic acid ethoxylated with 50 moles of ethylene oxide, monostearic acid ethoxylated with 100 moles of ethylene oxide, monostearic acid ethoxylated with 200 moles of ethylene oxide, monostearic acid ethoxylated with 300 moles of ethylene oxide or monostearic acid ethoxylated with 1000 moles of ethylene oxide.

Among the hydrogenated and ethoxylated castor oils which can be combined with the composition which is a subject matter of the present invention as hydrophilic surfactants, mention may be made of hydrogenated castor oil ethoxylated with 5 moles of ethylene oxide, hydrogenated castor oil ethoxylated with 7 moles of ethylene oxide, hydrogenated castor oil ethoxylated with 10 moles of ethylene oxide, hydrogenated castor oil ethoxylated with 20 moles of ethylene oxide, hydrogenated castor oil ethoxylated with 25 moles of ethylene oxide, hydrogenated castor oil ethoxylated with 30 moles of ethylene oxide, hydrogenated castor oil ethoxylated with 40 moles of ethylene oxide, hydrogenated castor oil ethoxylated with 45 moles of ethylene oxide, hydrogenated castor oil ethoxylated with 50 moles of ethylene oxide, hydrogenated castor oil ethoxylated with 60 moles of ethylene oxide, hydrogenated castor oil ethoxylated with 80 moles of ethylene oxide or hydrogenated castor oil ethoxylated with 100 moles of ethylene oxide.

The composition according to the invention optionally comprises from 0 to 20% of at least one coating adjuvant selected from diluents, plasticizers, antifoaming agents and disintegrants.

Among the diluents which can be combined with the composition which is a subject matter of the present invention, mention may be made of: lactose, sucrose, mannitol, sorbitol, xylose, xylitol, isomalt, talc, native starches, silica, silica dioxide and magnesium stearate.

Among the plasticizers which can be combined with the composition which is a subject matter of the present invention, mention may be made of: glycerol, polypropylene glycols, polyethylene glycols or their derivatives of condensation with a fatty acid or a fatty alcohol, stearic acid and its derivatives, acetylated monoglycerides, esters of citric acid, such as, for example, triethyl citrate, acetyl triethyl citrate or acetyl tributyl citrate, triacetin, sorbitol or dibutyl seccate.

Among the antifoaming agents which can be combined with the composition which is a subject matter of the present invention, mention may be made of silicone derivatives.

Among the disintegrants which can be combined with the composition which is a subject matter of the present invention, mention may be made of: cellulose derivatives, crospovidones, sodium croscarmelloses and sodium starch glycolate.

It should be noted that other adjuvants such as fragrances, flavorings, appetizing agents, colorants, pigments and antioxidants may be combined with the composition which is a subject matter of the present invention.

A subject of the present invention is also a controlled-release composition (C_{A}) comprising:

at least one lipid composition according to the invention, and

at least one active pharmaceutical, prophylactic or food substance.

This composition (CA) according to the invention will preferably be intended for oral administration in human beings or animals.

According to another preferred embodiment, the composition (CA) according to the invention will be in solid form.

Among the pharmaceutical active principles, mention may be made of nonsteroidal anti-inflammatories and antirheumatics (ketoprofen, ibuprofen, flurbiprofen, indomethacin, phenylbutazone, allopurinol, and the like), analgesics (paracetamol, phenacetin, aspirin, and the like), antitussives (codeine, codethyline, alimemazine, and the like), sterols (hydrocortisone, cortisone, progesterone, testosterone, triamcinolone, dexamethasone, betamethasone, paramethasone, fluocinolone, beclomethasone, and the like), barbiturates (barbital, allobarbital, phenobarbital, pentobarbital, amobarbital, and the like), antimicrobials (pefloxacin, sparfloxacin, and derivatives of the class of quinolones, tetracyclines, synergistins, metronidazole, and the like), medicines intended for the treatment of allergies, antiasthmatics, vitamins (vitamin A, vitamins B, vitamin C, vitamin E, vitamins of the D group, vitamin K), antispasmodics and antisecretory agents (omeprazole), cardiovascular agents and cerebral vasodilators (quinacainol, oxprenolol, propanolol, nicergoline, and the like), cerebroprotective agents, hepatoprotective agents, therapeutic agents for the gastrointestinal tract, vaccines, antihypertensives and cardioprotective agents, such as beta blockers and nitro derivatives.

Among the nutritional agents, mention may be made of the active principles usually used in the field of nutrition, such as bioactive lipids, water-soluble or water-dispersible trace element salts, water-soluble or liposoluble vitamins, prebiotics, probiotics, milk proteins and/or milk protein concentrates, plant or animal enzymes, amino acids, peptides, sugars, flavor enhancers, flavoring agents, botanical ingredients (vegetable extracts of ginger, of curcumin, of St. John's wort, of valerian, blueberry extracts, pomegranate extracts, Chlorella vulgaris extracts, artichoke extracts, hibiscus extracts).

Among the bioactive lipids, mention may be made of phytosterols, such as those extracted from vegetable oils, and more particularly extracts of sea-buckthorn oil, corn oil or soybean oil; phytosterol complexes, isolated from vegetable oils, such as, for example, cholestatin, composed of campesterol, stigmasterol and brassicasterol; phytostanols; carotenoids, which belong to the family of the terpenoids, extracted from algae, green plants, fungi or bacteria; polyunsaturated fatty acids of the omega-3 group, such as, for example, α-linolenic acid, eicosapentaenoic acid or docosahexanoic acid; polyunsaturated fatty acids of the omega-6 group, such as, for example, linoleic acid, γ-linolenic acid, acid Among the water-soluble or water-dispersible trace element salts used in ingestible solid forms coated with the coating composition which is a subject matter of the present invention, mention may be made of ferrous carbonate, ferrous chloride tetrahydrate, ferric chloride hexahydrate, ferrous citrate hexahydrate, ferrous fumarate, ferrous lactate tetrahydrate, ferrous sulfate monohydrate, ferrous sulfate heptahydrate, ferrous chelate of amino acids hydrate, iron glycine chelate; calcium iodate hexahydrate, anhydrous calcium iodate; sodium iodide, potassium iodide; cobalt acetate tetrahydrate, basic cobalt carbonate monohydrate, cobalt carbonate hexahydrate, cobalt sulfate heptahydrate, cobalt sulfate monohydrate, cobalt nitrate hexahydrate; cupric acetate monohydrate, basic copper carbonate monohydrate, cupric chloride dihydrate, copper methionate, cupric sulfate pentahydrate, cuprous chelate of amino acids hydrate, cuprous chelate of glycine hydrate, copper chelate of hydroxy analog of methionine; manganous carbonate, manganous chloride tetrahydrate, manganese hydrogen phosphate trihydrate, manganous sulfate tetrahydrate, manganous sulfate monohydrate, manganese chelate of amino acids hydrate, manganese chelate of glycine hydrate, manganese chelate of hydroxy analog of methionine; ammonium molybdate, sodium molybdate, sodium selenite, sodium selenate; the organic form of selenium produced by Saccharomyces cerevisiae, selenomethionine (inactivated selenium yeast), and the selenomethionine produced by Saccharomyces cerevisiae (inactivated selenium yeast).

Among the inorganic salts, mention may be made of the salts of metal cations, such as, for example, the sodium, potassium, calcium, magnesium, zinc, manganese, iron, copper, cobalt, silver, barium, zirconium and strontium cations, and of organic anions, such as, for example, an edible organic anion having at least one carboxylic acid functional group in the carboxylate form, selected from the elements of the group consisting of the anions derived from glycolic, citric, tartaric, salicylic, lactic, mandelic, ascorbic, pyruvic, fumaric, glycerophosphoric, retinoic, benzoic, kojic, malic, gluconic, galacturonic, propionic, heptanoic, 4-aminobenzoic, cinnamic, benzalmalonic, aspartic and glutamic acids.

Among the inorganic salts, mention may more particularly be made of zinc gluconate, calcium gluconate, manganese gluconate, copper gluconate, magnesium aspartate, calcium aspartate, calcium glycerophosphate, calcium, magnesium glycerophosphate.

Among the water-soluble or liposoluble vitamins, mention may be made of vitamin A, more particularly in its form of retinol, retinyl acetate, retinyl palmitate or β-carotene, vitamin D2, more particularly in its form of ergocalciferol or -hydroxycalciferol, vitamin D3, more particularly in its form of cholecalciferol, vitamin K, more particularly in its form of phylloquinone (phytomenadione) or menaquinone, vitamin B1, more particularly in its form of thiamine hydrochloride, thiamine mononitrate, thiamine monophosphate chloride or thiamine pyrophosphate chloride, vitamin B2, more particularly in its form of riboflavin or riboflavin 5′-phosphate sodium, vitamin B6, more particularly in its form of pyridoxine hydrochloride, pyridoxine 5′-phosphate or pyridoxal 5′-phosphate, vitamin B12, more particularly in its form of cyanocobalamin, hydroxocobalamin, 5′-deoxyadenosylcobalamin or methylcobalamin, vitamin C, more particularly in its form of L-ascorbic acid, sodium L-ascorbate, calcium L-ascorbate, potassium L-ascorbate, calcium salts of 6-palmitoyl-L-ascorbic acid or sodium ascorbyl monophosphate, pantothenic acid, more particularly in its form of calcium D-pantothenate, sodium D-pantothenate, dexpanthenol or pantethine, vitamin PP, more particularly in its form of nicotinic acid, niacin, nicotinamide or inositol hexanicotinate (inositol hexaniacinate), vitamin B9, more particularly in its form of folic acid or folates, more particularly in their form of pteroylmonoglutamic acid, calcium L-methylfolate or (65)-5-methyltetrahydrofolic acid in the form of glucosamine salt, vitamin H2, B7 or BW, more particularly in its form of biotin, choline, more particularly in its form of choline chloride, choline dihydrogen citrate or choline bitartrate, inositol, carnitine, more particularly in its form of L-carnitine or L-carnitine L-tartrate, or taurine.

Among the prebiotics, mention may be made of inulin, transgalactooligosaccharides, fructans and mannooligosaccharides.

Among the probiotics, mention may be made of the various strains of Saccharomyces cerevisiae, of Bacillus cereus var. toyoi, of Bacillus subtilis alone or in combination with Bacillus licheniformis, or else strains of Enteroccocus faecium, lactic acid bacteria and more particularly lactobacilli, bifidobacteria and streptococci. These strains of microorganisms are generally combined with a solid support, for example calcium carbonate, dextrose or sorbitol.

Among the proteins and/or protein concentrates, mention may be made of milk proteins resulting from milk cracking, such as colostrum in the form of a lyophilized or atomized powder, whey in the form of a powder, of fractions which are purified or enriched in IgG, in lactoferrin or in lactoperoxidase. Among the plant or animal enzymes, mention may be made of Promutase, superoxide dismutase (SOD), 3-phytase, 6-phytase, endo-1,4-β-glucanases, endo-1,4-β-xylanases, or also other enzymes which improve or promote digestion.

Among the peptides, mention may be made of avocado peptides, lupin peptides, quinoa peptides, maca peptides, fermented or unfermented soybean peptides, rice peptides, peptides present in Acacia macrostachya seed extract or peptides present in passionflower seed extracts.

Among the amino acids, mention may be made of alanine, arginine, asparagine, aspartic acid, cysteine, glutamic acid, glutamine, glycine, histidine, isoleucine, leucine, lysine, methionine, phenylalanine, proline, hydroxyproline, pyrrolysine, selenocysteine, serine, threonine, tryptophan, tyrosine, valine, sarcosine or ornithine.

Among the sugars, mention may be made of water-soluble polysaccharides, or sugars of lower molecular weight, such as oligosaccharides or mono- or disaccharides, such as, for example, glucose, lactose or dextrose.

Among the flavor enhancers, mention may be made of glutamates, such as, for example, glutamic acid, monosodium glutamate, monopotassium glutamate, calcium diglutamate, ammonium glutamate or magnesium diglutamate; guanylates, such as, for example, guanylic acid (guanosine monophosphate), disodium guanylate, dipotassium guanylate or calcium guanylate, inosinates, such as, for example, inosinic acid, disodium inosinate, dipotassium inosinate or calcium inosinate, or also intense sweeteners, such as Stevia extracts or rebaudiosides.

A subject of the present invention is also the use of a lipid composition according to the invention for the encapsulation of an active pharmaceutical, therapeutic, prophylactic, or food substance for human beings or animals in a galenical formulation.

For the purposes of the present invention, the term “encapsulation” is understood to mean an operation making it possible to bring together a lipid composition and one or more active principles. The active agent or agents may be located inside a shell formed by this “cooled” lipid composition or dispersed within the latter.

Lastly, a final subject of the present invention is a process for manufacturing a galenical formulation comprising a composition (CA) according to the invention, comprising at least:

a) a step of preparing the lipid composition according to the invention,
b) a step of mixing and encapsulating an active pharmaceutical, prophylactic or food substance with the lipid composition prepared in step a) so as to obtain a composition (C_{A}) according to to the invention, and
c) a step of galenically forming the composition (C_{A}) prepared in step b), involving mechanical stress.

The term “mechanical stress” is understood to mean a tension or pressure which acts on a material and which may change the shape or properties of said material. In the context of the invention, this is a stress exerted on the composition CA during its use for preparing the final galenical form (step c).

Still in the context of the invention, it is noted that the release profile of the active substance in the final galenical form is similar or not greatly different from the release profile of the active substance in the composition CA. Specifically, it is because of the lipid composition according to the invention that a resistance to mechanical stress and hence little or no change in the release profile are observed.

Among the encapsulation processes used in step b), mention may be made of prilling, spray chilling or spray cooling, spray congealing, hot melt coating, hot melt extrusion, melt granulation, pelletization, spheronization, or thermogranulation, etc. (insert the French translation where possible).

The term “prilling” denotes a process of coating by dissolving or dispersing the active principle or principles in a molten lipid composition, and then spraying this into ambient or cooled air, or into a cooled liquid.

The “spray chilling”, “spray cooling” and “spray congealing” processes are particular processes of the prilling process.

The term “hot melt coating” denotes a process of coating by spraying a molten lipid composition onto a solid particle consisting of the active agent or a mixture of active agents. Depending on the case, the manufacturing process according to the invention may have one or more of the following features:

the mixing and encapsulation step b) comprises a first sub-step of heating the lipid composition prepared in step a) to a temperature 10 to 15° C. greater than the highest melting point of the various ingredients of said lipid composition so as to melt said lipid composition, a second sub-step of mixing the molten lipid composition with the active substance in dispersed or molten form, and a third sub-step of spraying the composition obtained in the second sub-step into ambient or cooled air or into a cooled liquid in order to obtain solidified particles of composition (C_{A}); the term “ambient air” is understood to mean air at an ambient temperature generally in the vicinity of 25° C.; the term “cooled air” or “cooled liquid” is understood to mean air or liquid at a temperature of less than ambient temperature, in other words of less than 25° C.;

the galenical forming step involving mechanical stress is selected from processes of rendering into solid oral dosage forms, and for example compression, forming into hard capsules (capsule filling/molding), compacting (compaction, roller compaction), packaging, placing into sachets, forming into sticks (stick and sachet filling), extrusion, granulation, and pelletization; these techniques can result in the manufacture of the following forms (non-exhaustive): oral powder, beads, hard capsules (capsules), granules, sugar-coated granules, pearls, pellets, spheres (spherules), tablets, sachets, sticks, chews, gums, chewable tablets, chewing gums.

The lipid composition according to the invention makes it possible to form an active substance by various coating technologies, unexpectedly inducing a stability of the release profile of the active substance thus coated compared to the release profile of the active substance the galenical form of which has not been obtained by a process comprising a step of mechanical stress.

In other words, the subject matter of the present invention makes it possible to maintain the release profile of the active substance initially sought regardless of the downstream galenical forming process performed, and regardless of the mechanical stress applied to render the active agent into the finished product form (mixing, compaction, forming into hard capsules, forming into a stick, tableting, etc.).

For the purposes of the present invention, the term “forming according to technologies for coating active agents” is understood to mean any primary technology or any process making it possible to confer a physical form, preferably a form which is solid at ambient temperature, upon the composition comprising said active agent.

The examples that follow illustrate the invention without, however, limiting it.

### DESCRIPTION OF THE PREFERRED EMBODIMENTS

### Examples

The qualification of the encapsulation compositions as described above inducing properties of resistance to the mechanical stresses of the downstream galenical steps is established by setting up analyses as described below.

An active agent is chosen as a principal model active agent. Note that the term “active agent” is understood to mean an active substance. This active substance is caffeine, with an average solubility at ambient temperature (25° C.) in water of 20-25 g/I. Molecular formula: C8H10N4O2; molecular weight: 194.194 g/mol.

This active agent is encapsulated with the compositions which are the subject matter of the present invention at a rate of 20% to 30% by rotating disk prilling technology. Rotating disk prilling technology: process for preparing composition M1

For each test, a waxy liquid dispersion is prepared. This waxy liquid dispersion consists of:
the composition which is a subject matter of the present invention, in molten form;
the model active agent in dispersed solid form. In the general case, it should be remembered that an active agent may be present in dispersed or liquid form depending on the value of its melting point.

For this, a “pre-dispersion”—the result of the melting and/or mixing in the liquid state of the various elements of the composition which is a subject matter of the present invention—is produced beforehand: mixture preparation M0.

The temperature at which this pre-dispersion, and then the reconstituted dispersion, is kept should be adjusted so as to be 10 to 15° C. greater than the highest melting point of the various ingredients of the composition. Before starting the manufacturing process, the active agent is added, dispersed in this case, with mechanical stirring while maintaining the previously established temperature.

The rotating disk prilling process is then performed. The dispersion is conveyed by means of heat-insulated pipes to a spray nozzle located in a space/tower resulting, after spraying, to the creation of fine droplets. These fine droplets are then solidified in a stream of cold ambient air, leading to the formation of small spherical beads characterized in that 50% by volume of these beads have a diameter of between 300 and 500 μm. It will be noted that the particle size profile of the beads is measured by virtue of a Mastersizer 3000 laser particle sizer from Malvern, used in the dry route, at a pressure of 1 bar.

Silicon dioxide or another flow/anti-stick agent can optionally be added beforehand to the microbeads thus produced in order to facilitate later handling thereof.

Likewise, sieving on a 500 μm sieve may be performed in order to remove any undesired agglomerate/residue having a particle diameter greater than or equal to 500 μm.

The microbeads thus obtained are then incorporated into a mixture of excipients of grades compatible with a tablet-type galenical forming process. The tablet form is chosen as model final galenical forming process since it represents one of the most extreme cases for induced mechanical stress.

The tableting is carried out on an instrumented Dott Bonapace reciprocating single-punch press or an instrumented Riva Piccola rotary 8-punch press with application of a compression force which can vary between 5 and 20 kN; 500 mg tablets with breaking strengths of between 80 and 120 N are thus manufactured.

Demonstration of the technical effect:

The release profile of the active agent, coated in the composition which is a subject matter of the present invention and then incorporated into a tablet format, is established by means of an Erweka dissolution tester following the recommendations of the European Pharmacopoeia, version 7.3, paragraph 2.9.3.

The dissolution medium mainly chosen is a pH 7.2 phosphate buffer maintained at a temperature of 37° C. Samples are taken periodically for up to 6 hours. The samples are then analyzed by reversed-phase HPLC assay with UV detection in order to determine the amount of active agent present in each sample and thus to establish a dissolution profile per sample to be evaluated.

The dissolution profiles, from composition to composition or comparison before/after application of a mechanical stress, are compared in order to determine the yes/no difference of two profiles with each other or of one profile with respect to a control profile.

Two profiles are thus judged to be similar or not very different, and hence the composition is judged to improve the resistance to mechanical stress, when:
The time-to-time difference, the calculation of which is as follows:

abs[% released(Txi)tab−% released(Txi)μbeads]≤y %
where:
% released(Txi)tab=% of active agent released at the sampling point Txi when the formulation has undergone a tableting process.
% released(Txi)μbeads=% of active agent released at the sampling point Txi when the formulation has not undergone mechanical stress
and in terms of value y is less than or equal to 30+/−2% for up to 2 hours of dissolution time and less than or equal to 20+/−2% over the remaining evaluation time.
The profile can be judged to be similar when the difference throughout the dissolution test is less than or equal to 15%.
The rate of change between the values before and after application of a mechanical stress is less than or equal to 30% as from 3 hours of dissolution time. The calculation is as follows:

$$\frac{\% released ( Txi ) tab - \% released ( Txi ) μbeads}{\% released ( Txi ) μbeads} \leq 30 \% as from 180 min$$

where:

Txi>=180 minutes
% released(Txi)tab=% of active agent released at the sampling point Txi when the formulation has undergone a tableting process(involving a compression step).
% released(Txi)μbeads=% of active agent released at the sampling point Txi when the formulation has not undergone mechanical stress

### Example 1

In this example, lipid compositions containing as component A the same beeswax, the same amount of polysorbate 80, and the same proportion of various lipophilic surfactants as component B. The constitution of the lipid compositions prepared is recorded in table 1 below. These lipid compositions are used for the coating according to the prilling process as described above with caffeine as the active ingredient. The aim of this example is to show the validity of the combination of beeswax with certain lipophilic surfactants for the coating by the prilling process of the active agent caffeine, by observing the maintenance or non-maintenance of a release profile over the course of time, so as to possibly not consider as appropriate certain combinations of beeswax and lipophilic surfactants before they are subjected to a compression step.

TABLE 1

| Identification of | | | |
|---|---|---|---|
| the composition | CL 1 | CL 2′ | CL 3′ |
| Beeswax | 80.0% | 80.0% | 80.0% |
| Sorbitan stearate | 19.6% | | |
| (Montane 60 SEPPIC) | | | |
| Glycerol distearate | | 19.6% | |
| (EP name) | | | |
| Glycerol Monostearate | | | 19.6% |
| (EP name) | | | |
| Polysorbate 80 | 0.4% | 0.4% | 0.4% |
| (Montanox 80 SEPPIC) | | | |
| | 100% | 100% | 100% |

The active agent is dispersed at 20% in the composition. The prilling process is implemented. Microbeads having a median diameter of between 350 and 400 μm are obtained.

These microbeads are stabilized at ambient temperature, protected from light, for a minimal duration of 28 months.

The dissolution profiles of the microbeads after the manufacturing process or after a minimal shelf life of 28 months are studied. Various sampling times are realized; the comparison of the profile is carried out at the 120 minute point.

Table 2 below gives percentage values of caffeine released at the 120 minute sampling point:

TABLE 2

| | | CL 1′ - at a | | CL 2′ - at a | | CL 3′ - at a |
|---|---|---|---|---|---|---|
| | | minimum of | | minimum of | | minimum of |
| | CL1′ - at T0 | T28 months | CL2′ - at T0 | T28 months | CL3′ - at T0 | T28 months |
| Identification | from the | from the | from the | from the | from the | from the |
| of the | obtaining | obtaining | obtaining | obtaining | obtaining | obtaining |
| composition // | of the | of the | of the | of the | of the | of the |
| sampling time | microbeads | microbeads | microbeads | microbeads | microbeads | microbeads |
| 120 minutes | 71% | 75% | 73% | 64% | 100% | 75% |

Table 3 below gives the values of the time-to-time differences* at the 120 minute sampling point:

TABLE 3

| Identification of | CL1′ - | CL2′ - | CL3′ - |
|---|---|---|---|
| the composition // | time-to- | time-to- | time-to- |
| sampling time | time difference | time difference | time difference |
| 120 minutes | 4% | 9% | 25% |

(*): a specific definition of the time-to-time difference should be noted here, in the case of a comparison of the release profile at T0 and after a period of stabilization. Therefore, the time-to-time difference is redefined here as resulting from the following calculation:

abs[% released(120x′i)μbeads−% released(120xi)μbeads]y %
where:
% released (120x′i)μbeads=% of active agent released at the 120 minute sampling point, when the formulation has been stored for a defined duration at ambient temperature.
released (120xi)μbeads=% of active agent released at the 120 minute sampling point, when the formulation has not been stored, i.e. the value at T0 from the production of the microbead.

Two profiles are judged to be of the same order when the time-to-time difference in terms of value y is less than or equal to 20+/−2% at the sampling point considered.

The profile can be judged to be similar when the time-to-time difference is less than or equal to 15%.

For this example, it can be observed that the compositions CL1′ and CL2′ make it possible to maintain over time the release profile of the active agent caffeine contained in the microbeads resulting from the prilling process. The composition CL3′ containing glycerol monostearate as component B can already be discounted.

### Example 2

In this example, lipid compositions containing as component A waxes and oils from various sources which are used for the coating by the prilling process of the active agent caffeine as described above. The aim of this example is to demonstrate the specificity of beeswax, combined with the other components, for making it possible to maintain a similar/not greatly different, or otherwise, release profile of the coated active agent after the galenical forming process/following mechanical stress.

The following compositions are thus used in parallel (table 4):

TABLE 4

| Identification of | | | | |
|---|---|---|---|---|
| the composition | CL 1 | CL 2 | CL 3 | CL 4 |
| Beeswax | 80.0% | | | |
| Palm oil | | | | 80.0% |
| Candelilla wax | | 80.0% | | |
| Carnauba wax | | | 80.0% | |
| Sorbitan stearate | 19.6% | 19.6% | 19.6% | 19.6% |
| (Montane 60 SEPPIC) | | | | |
| Polysorbate 80 | 0.4% | 0.4% | 0.4% | 0.4% |
| (Montanox 80 SEPPIC) | | | | |
| | 100% | 100% | 100% | 100% |

The active agent is dispersed at 20% in the composition. The prilling process is implemented. Microbeads having a median diameter of between 350 and 400 μm are obtained.

These microbeads are then introduced into a mixture for tablets. 500 mg tablets having a diameter of 11 mm are thus produced according to the following composition: for 40% by weight of microbeads, 27% by weight of microcrystalline cellulose, 29% by weight of calcium hydrogen phosphate dihydrate, 3% by weight of crospovidone and 1% by weight of magnesium stearate are added.

The dissolution profiles of the microbeads before and after compression process are studied. The sampling times are as follows: 60, 120, 180, 240, 300 and 360 minutes.

Table 5 below gives the percentage values of caffeine released as a function of time:

TABLE 5

| | CL1 - | | CL2 - | | CL3 - | | CL4 - | |
|---|---|---|---|---|---|---|---|---|
| Identification | before | | before | | before | | before | |
| of the | galenical | CL 1 - | galenical | CL 2 - | galenical | CL 3 - | galenical | CL 4 - |
| composition // | forming | after | forming | after | forming | after | forming | after |
| sampling time | process | tableting | process | tableting | process | tableting | process | tableting |
| 60 minutes | 40% | 49% | 3% | 24% | 6% | 26% | 9% | 23% |
| 120 minutes | 71% | 73% | 5% | 29% | 8% | 32% | 10% | 28% |
| 180 minutes | 87% | 86% | 6% | 33% | 9% | 36% | 10% | 30% |
| 240 minutes | >90% | >90% | 7% | 36% | 11% | 39% | 11% | 32% |
| 300 minutes | >90% | >90% | 7% | 38% | 12% | 42% | 11% | 33% |
| 360 minutes | >90% | >90% | 8% | 40% | 12% | 44% | 11% | 34% |

Table 6 below gives the values of the time-to-time differences and the rates of change at 180 minutes and beyond:

For this example, it can be seen that only composition CL1 allows an improvement in the resistance to mechanical stress, this stress being illustrated here by a tablet-type galenical forming process.

### Example 3

In this example, lipid compositions containing as component A a ratio of beeswax and candelilla wax which are used for the coating by prilling process of the active agent caffeine. The aim of this example is to demonstrate the possibility of mixing beeswax with another lipid compound up to a certain ratio, combined with the other components, while still making it possible to maintain a similar/not greatly different, or otherwise, release profile of the coated active agent after the galenical forming process/following mechanical stress.

The following compositions are thus used in parallel:

TABLE 7

| Identification of | | | |
|---|---|---|---|
| the composition | CL 1 | CL 5 | CL 6 |
| Beeswax | 80.0% | 76.0% | 68.0% |
| Candelilla wax | | 4.0% | 12.0% |
| Sorbitan stearate | 19.6% | 19.6% | 19.6% |
| (Montane 60 SEPPIC) | | | |
| Polysorbate 80 | 0.4% | 0.4% | 0.4% |
| (Montanox 80 SEPPIC) | | | |
| | 100% | 100% | 100% |

The active agent is dispersed at 20% in the composition. The prilling process is implemented. Microbeads having a median diameter of between 350 and 400 μm are obtained.

These microbeads are then introduced into a mixture for tablets prepared according to the procedure described in example 2. 500 mg tablets having a diameter of 11 mm are thus produced.

The dissolution profiles of the microbeads before and after compression process are studied. The sampling times are as follows: 60, 120, 180, 240, 300 and 360 minutes.

The table below gives the percentage values of caffeine released as a function of time:

TABLE 8

| | CL1 - | | CL5 - | | CL6 - | |
|---|---|---|---|---|---|---|
| Identification | before | | before | | before | |
| of the | galenical | CL 1 - | galenical | CL 5 - | galenical | CL6 - |
| composition // | forming | after | forming | after | forming | after |
| sampling time | process | tableting | process | tableting | process | tableting |
| 60 minutes | 40% | 49% | 27% | 59% | 10% | 48% |
| 120 minutes | 71% | 73% | 52% | 79% | 22% | 67% |
| 180 minutes | 87% | 86% | 71% | 89% | 35% | 77% |
| 240 minutes | >90% | >90% | 83% | >90% | 46% | 84% |
| 300 minutes | >90% | >90% | 90% | >90% | 56% | 87% |
| 360 minutes | >90% | >90% | >90% | >90% | 64% | 90% |

The table below gives the values of the time-to-time differences and the rates of change at 180 minutes and beyond:

TABLE 9

| Identification | CL1 - | | CL5- | | CL6 - | |
|---|---|---|---|---|---|---|
| of the | time-to- | CL 1 - | time-to- | CL 5 - | time-to- | CL6 - |
| composition // | time | rate of | time | rate of | time | rate of |
| sampling time | difference | change | difference | change | difference | change |
| 60 minutes | 9% | | 31% | | 38% | |
| 120 minutes | 2% | | 26% | | 45% | |
| 180 minutes | 0% | 0% | 19% | 26% | 43% | >100% |
| 240 minutes | N/A | N/A | N/A | N/A | 37% | 80% |
| 300 minutes | N/A | N/A | N/A | N/A | 31% | 56% |
| 360 minutes | N/A | N/A | N/A | N/A | 25% | 39% |

In this example, compositions CL1 and CL5 allow an improvement in the resistance to mechanical stress, with a similar profile for composition CL1 and a not greatly different profile for composition CL5.

### Example 4

In this example, lipid compositions having a component A (beeswax) and a component B (hydrophobic surfactant of sorbitan ester type) which are used for the coating by prilling process of the active agent caffeine.

The aim of this example is to demonstrate the possibility of mixing component A and component B, for a certain ratio, with regard to maintaining a similar/not greatly different, or otherwise, release profile of the coated active agent after the galenical forming process/following mechanical stress.

The following compositions are thus used in parallel:

TABLE 10

| Identification of the | | | |
|---|---|---|---|
| composition | CL7 | CL8 | CL9 |
| Beeswax | 100.0% | 80.0% | 60.0% |
| Sorbitan stearate | | 20.0% | 40.0% |
| (Montane 60 SEPPIC) | | | |
| | 100% | 100% | 100% |

The active agent is dispersed at 20% in the composition. The prilling process is implemented. Microbeads having a median diameter of between 350 and 400 μm are obtained.

These microbeads are then introduced into a mixture for tablets prepared according to the procedure described in example 2. 500 mg tablets having a diameter of 11 mm are thus produced.

The dissolution profiles of the microbeads before and after compression process are studied. The sampling times are as follows: 30, 60, 120, 180, 240, 300 and 360 minutes.

Table 11 below gives the percentage values of caffeine released as a function of time:

The table below gives the values of the time-to-time differences and the rates of change at 180 minutes and beyond:

In this example, compositions CL8 and CL9 allow an improvement in the resistance to mechanical stress. Via composition CL7, it is observed that a minimal amount of component B is required.

1. A lipid composition comprising, per 100% of weight:
from 40% to 99.9% by weight of a component comprising, per 100% of weight, from 90% to 100% by weight of beeswax and up to 10% by weight of at least one other lipid excipient,
from 0.1% to 60% by weight of at least one lipophilic surfactant selected from polyethoxylated fatty acids, esters of fatty diacids and of polyethylene glycols, esters of polyglycerol and of fatty acids, esters of propylene glycol and of fatty acids, mixtures of esters of propylene glycol and of esters of glycerol, fatty acid diglycerides, sterols and derivatives of sterol, esters of fatty acids and of sorbitan, esters of sorbitan, of polyethylene glycols and of fatty acids, ethers of polyethylene glycols and of alkyl, sucrose esters, and polyoxyethylene-polyoxypropylene block copolymers.

2. The lipid composition as claimed in claim 1, wherein the lipid excipient is selected from animal waxes, vegetable waxes, mineral waxes, synthetic waxes or hydrogenated vegetable oils.

3. The lipid composition as claimed in claim 1, wherein the lipophilic surfactant is selected from esters of fatty acids and of sugars.

4. The lipid composition as claimed in claim 1, wherein the lipophilic surfactant is a lipophilic surfactant from the family of the sorbitan esters.

5. The lipid composition as claimed in claim 1, wherein said composition comprises from 0 to 20% of one or more hydrophilic surfactants.

6. The lipid composition as claimed in claim 5, wherein the hydrophilic surfactant or surfactants is/are selected from soya lecithin, ethoxylated sorbitan esters, polyethoxylated alcohols, polyethoxylated acids, polyglycerol esters, glucose ethers and block copolymers of ethylene oxide and of propylene oxide.

7. The lipid composition as claimed in claim 5, wherein the hydrophobic surfactant is a sorbitan ester and the hydrophilic surfactant is selected from polyethoxylated sorbitan esters.

8. The lipid composition as claimed in claim 7, wherein said composition comprises:
75% beeswax,
20% sorbitan stearate, and
5% sorbitan oleate polyethoxylated with 20 moles of ethylene oxide (or polysorbate 80).

9. The lipid composition as claimed in claim 7, wherein said composition comprises:
50% beeswax,
45% sorbitan stearate, and
5% sorbitan oleate polyethoxylated with 20 moles of ethylene oxide (or polysorbate 80).

10. The lipid composition as claimed in claim 1, wherein said composition comprises from 0 to 20% of at least one coating adjuvant.

11. The lipid composition as claimed in claim 8, wherein the coating adjuvant or adjuvants is/are selected from diluents, flavorings, appetizing agents, colorants, antioxidants, plasticizers, antifoaming agents and disintegrants.

12. A controlled-release composition (CA) comprising:
at least one composition as defined in claim 1, and
at least one active pharmaceutical, prophylactic or food substance.

13. The composition (CA) as claimed in claim 12, wherein the composition is suitable for oral administration in human beings or animals.

14. The composition (CA) as claimed in claim 12, wherein the composition is in solid form.

15. A method for the encapsulation of an active pharmaceutical, therapeutic, prophylactic, or food substance for human beings or animals in a galenical formulation, comprising providing the lipid composition of claim 1, and encapsulating the active pharmaceutical, therapeutic, prophylactic, or food substance within the lipid composition.

16. A process for manufacturing a galenical formulation comprising a composition (CA) as claimed in claim 12, comprising at least:
a) a step of preparing the lipid composition,
b) a step of mixing and encapsulating an active pharmaceutical, prophylactic or food substance with the lipid composition prepared in step a) so as to obtain a composition (CA) as claimed in claim 12, and
c) a step of galenically forming the composition (CA) prepared in step b), involving mechanical stress.

17. The manufacturing process as claimed in claim 16, wherein the mixing and encapsulation step b) comprises:
a first sub-step of heating the lipid composition prepared in step a) to a temperature 10 to 15° C. greater than the highest melting point of the various ingredients of said lipid composition so as to melt said lipid composition,
a second sub-step of mixing the molten lipid composition with the active substance in dispersed or molten form, and
a third sub-step of spraying the composition obtained in the second sub-step into ambient or cooled air or into a cooled liquid in order to obtain solidified particles of composition (CA).

18. The manufacturing process as claimed in claim 16, wherein the galenical forming step involving mechanical stress is selected from compression, forming into hard capsules, compacting, packaging, placing into sachets, forming into sticks, extrusion, granulation, and pelletization.

19. The lipid composition as claimed in claim 2, wherein the lipophilic surfactant is selected from esters of fatty acids and of sugars.

20. The lipid composition as claimed in claim 2, wherein said composition comprises from 0 to 20% of one or more hydrophilic surfactants.
